(19) United States
(12) Patent Application Publication
(10) Pub. No.: US 2008/0300560 A1
(43) Pub. Date: Dec. 4, 2008

(54) Absorbent Article

(75) Inventors: Elinor Magnusson, Molnlycke (SE); Carolyn Berland, Molndal (SE)

Correspondence Address: BUCHANAN, INGERSOLL & ROONEY PC, POST OFFICE BOX 1404, ALEXANDRIA, VA, 22313-1404, US

(73) Assignee: SCA HYGIENE PRODUCTS AB, Goteborg (SE)

(21) Appl. No.: 12/093,424

(22) PCT Filed: Nov. 30, 2005
(86) PCT No.: PCT/SE05/01804

§ 371 Date: May 12, 2008

(51) Int. Cl. A61F 13/15

(52) U.S. Cl. 604/367; 604/378; 604/385.23; 604/385.01; 604/386

(57) ABSTRACT

An absorbent article, such as a diaper or an incontinence protection product, which has a longitudinal direction and a transverse direction, a front waist portion, a rear waist portion and a crotch portion which is arranged between the front and rear waist portions and includes a liquid-permeable outer layer and a liquid-impermeable outer layer and also an absorption body which is enclosed between the outer layers, a front edge which extends in the transverse direction and a rear edge which extends in the transverse direction, with the article having a wiping area which is arranged on the liquid-permeable outer layer, with the wiping area being arranged outside the crotch area on the front waist portion and including an adsorptive agent for taking up and binding microorganisms and proteins.

### TECHNICAL FIELD

The invention relates to an absorbent article, such as a diaper or an incontinence protection product, which has a longitudinal direction and a transverse direction, a front waist portion, a rear waist portion and a crotch portion arranged between the front waist portion and the rear waist portion, and which comprises a liquid-permeable outer layer and a liquid-impermeable outer layer, and also an absorption body enclosed between the outer layers, a front edge which extends in the transverse direction and a rear edge which extends in the transverse direction, with the article exhibiting a wiping area which is arranged on the liquid-permeable outer layer.

### BACKGROUND

Diapers and incontinence protection products are understood as meaning absorbent articles which are designed to be worn like underpants and which, during use, cover the lower part of a user's trunk. Examples of these articles are diapers which are fastened together at the sides when being put on, and pant diapers and different types of belted products, i.e. diapers and incontinence protection products which are kept in place with the aid of a waist-encircling belt. A common feature of the intended type of absorbent articles is that they comprise a crotch portion and also a front waist portion and a rear waist portion.

Almost without exception, baby's diapers and incontinence protection products are changed by a carer, for example one of the child's parents in the case of baby's diapers or nursing staff or a relative in the case of incontinence protection products.

When a diaper or the like is being changed, it happens not infrequently that the lower abdomen of the user is contaminated with faecal matter, with careful cleaning of the lower abdomen being required before a new article can be applied to the user.

It is usual for the faecal matter to be removed in more than one stage, with the first stage being a rough cleaning of the contaminated part of the skin in which as much as possible of the faecal matter which has adhered to the user is wiped off. After that, a more careful cleaning or washing of the soiled part is carried out before a new absorbent article can be placed on the user.

U.S. Pat. No. 5,607,760 describes an absorbent article whose surface has been treated with a special lotion which, during use, is transferred to the user's skin and thereby reduces the risk of faecal matter adhering to the user's skin. The lotion also makes it easier to wipe off faecal matter which has nevertheless adhered to the skin. A problem associated with applying a lotion or other similar agents to the liquid-receiving surface of an absorbent article is that the agent interferes with the absorptive ability of the article. On the one hand, the ability of the article to admit liquid is reduced as a result of the lotion blocking pores in the liquid-receiving outer layer and, on the other hand, the lotion migrates into the article and, in connection with this, can have a negative influence on the absorptive ability of the article. The lotion can also entrain bacteria and other contaminants to the user's skin.

Another method of facilitating cleaning of the lower abdomen of the user is to provide the absorbent article with special reception areas or pockets for faecal matter. The aim of these articles is to retain the faecal matter in the absorbent article to the greatest extent possible and thereby reduce the risk of soiling the user's skin.

U.S. Pat. No. 6,648,865 describes an absorbent article in which low-viscosity faecal matter is separated from faecal matter of higher viscosity, while WO 00/28929 describes an absorbent article which has a special reception area for faecal matter which is arranged in the rear part of the article. The reception area comprises a reception material which exhibits relatively large apertures through which faecal matter can penetrate into the article and in which faecal matter readily becomes caught.

Notwithstanding the efforts which have been made with regard to reducing faecal soiling when using absorbent articles such as diapers and incontinence protection products, there nevertheless always remains a certain need for cleaning when changing an absorbent article which is contaminated with faecal matter.

As has previously been mentioned, such cleaning takes place in two stages, involving a first wiping stage and a second cleaning or washing stage.

Many carers employ the used absorbent article for the first wiping stage. This is done by the outside of the article being grasped by the front part, which is not contaminated with faeces, after which the inside of the front part of the article is used for wiping off faecal matter from the lower abdomen of the wearer and collecting the faecal matter in the used absorbent article. The article is then folded or rolled together and sealed in an expedient manner before being discarded.

A problem with faecal soiling is that the user's skin becomes contaminated with harmful bacteria which give rise to an obnoxious odour and skin irritation. Even if the initial wiping stage appears to be sufficient for removing the faecal matter, there is therefore a need for a more careful cleaning of the skin after the first wiping stage. This is naturally time-consuming and irritating both for the carer and the user of the absorbent article. In addition, it is desirable to reduce the need for using soap and other cleaning agents since they dry out the skin and can cause skin irritation. For this reason, it is frequently customary to lubricate the skin with some type of lotion or ointment after cleaning.

Furthermore, long-term use of a diaper or an incontinence article can result in the quantity of microorganisms on the user's skin being increased even if there is no faecal matter in the diaper or the incontinence article. It is therefore desirable to clean the skin when changing diapers or incontinence articles, so as to reduce the number of microorganisms on the skin, even when the diaper only contains urine. It is common for the carer to omit this cleaning stage in order to save time and minimize the use of soap and other agents on sensitive skin.

### OBJECTS AND SUMMARY

One object of the invention is therefore to provide an absorbent article which reduces the need for supplementary cleaning of a faeces-soiled skin part.

Another object of the invention is to provide an absorbent article whose function in wiping off faecal matter is improved but whose liquid acquisition ability and absorption capacity are essentially unaffected.

The present invention has now resulted in an absorbent article, such as a diaper or an incontinence protection product of the type mentioned in the introduction, which essentially removes the disadvantages associated with previously known articles of this nature.

The article is intended to be worn like underpants around the lower part of the trunk of a user and has a longitudinal direction and a transverse direction, a front waist portion, a rear waist portion and a crotch portion arranged between the front waist portion and the rear waist portion, and also comprises a liquid-permeable outer layer and a liquid-impermeable outer layer, and an absorption body which is enclosed between the outer layers, a front edge which extends in the transverse direction and a rear edge which extends in the transverse direction. A wiping area, which is suitable for wiping off faecal matter, is arranged on the water-permeable outer layer.

An article has the wiping area being arranged outside the crotch area of the article on the front waist portion and the wiping area includes adsorptive agents for taking up and binding microorganisms and proteins.

The crotch portion is understood here as being the area of the absorbent article which is expected to be the first to be wetted by body liquid. This area must have a high liquid acquisition capacity and should therefore be free from different types of agents which can prevent or impair the flow of liquid into the product. The crotch area is usually regarded as extending over approx. 50% of an absorbent article's total length between the front edge and the rear edge and can be slightly displaced forwards or backwards depending on the design of the article.

The waist portions are the areas of the absorbent article which are located at the ends of the article and which are located closest to the waist of the user when the article is being worn. These areas are normally not expected to be primarily wetted by body liquids or other secretions and a high admission ability is not, therefore, a critical property for the surface material in the waist portions.

In order to be able to adsorb and bind microorganisms and proteins to the wiping area, it is necessary that the adsorptive agent is firmly united with the absorbent article and is not transferred to the wearer's skin during use. Nor should the agent migrate into the article either during storage and transport of the article or when the article is being used. An agent which does not remain on the wiping area loses its function of binding bacteria and proteins and can in addition have a negative influence on the absorption properties of the absorbent article. Another disadvantage associated with an adsorptive agent which is transferred to the wearer's skin is that it can also transfer bacteria to skin parts which were not previously contaminated.

The agent for taking up and binding bacteria and proteins comprises a material which has been grafted with positively charged and/or hydrophobic groups.

Hydrophobic plastics, i.e. plastics, such as polyolefins, polystyrene and polytetrafluoroethylene, having a wetting angle which is at least 90°, have a certain ability to adsorb and chemically bind microorganisms and proteins. This ability can be improved by means of the original material being treated for the purpose of increasing its adsorptive ability.

A material which exhibits a combination of hydrophobic groups and cationic groups has a particularly good ability to bind bacteria and proteins. A way to produce such a material is to form a hydrophobic main chain onto which cationic groups are grafted. In this connection, the main chain is expediently formed by polymerizing a monomer which contains two or more ethylenically unsaturated units but does not contain any polar groups. Examples of suitable monomers are olefins (ethylene, propylene, etc.), aromatic vinyl compounds such as styrene, or other monomers consisting of C and H atoms. Monomers containing cationic groups, such as allylamine, vinylamine, vinylpyrridine, alkylaminoethyl (meth)acrylate and acrylamide, and their derivatives and salts), have been grafted onto the unsaturated units. It is of course possible, as an alternative, to use a polymer with a main chain containing the cationic groups and being grafted with hydrophobic side chains. The same monomers as specified above can be used in this connection.

Another example of a useful material having hydrophobic and cationic groups is described in WO 2004/062703. The material which is described is an electrophilic fibre material in which the fibres can be synthetic or cellulose-based and exhibit functional groups which interact with anionic groups or amino groups on the cell walls of microorganisms. Examples of these functional groups in microorganisms are carboxyl groups, amino groups, hydroxyl groups and the like, particularly (anionic) carboxyl groups and phosphoryl groups as in proteins, glycoproteins and certain polysaccharides, and amino groups as in proteins. The carboxyl and phosphoryl groups exhibit electrostatic interaction with cationic groups on the modified fibres while amino groups can interact with aldehyde groups, for example, on the modified fibres.

Examples of electropositive groups which can be used for modifying the fibres are common cationic groups, such as ammonium groups, phosphonium groups and sulphonium groups, and certain metal-containing groups. Examples of uncharged groups having internal charge separation include carbonyl groups such as aldehydes, ketones and the like. As an example, the internal charge separation in an aldehyde can be illustrated by the variant formula R—HC⁺—O— of the conventional neutral formula R—HC═O. The partially charged carbon atom can react with nucleophiles such as hydroxyl groups and amino groups.

According to another embodiment of the invention, the agent for taking up and binding bacteria and proteins comprises a wax having a melting point which is higher than 45° C. Such a wax can be applied in the form of a continuous coating or as a patterned coating, for example a dot or screen pattern on the wiping area. Due to the wax having a high melting point, it does not melt because of the body temperature but instead remains on the wiping area when the absorbent article is used and does not pass over onto the skin of the user or migrate into the article.

Examples of waxes which can be used are ceresin, paraffins, microcrystalline paraffin or petrolatum waxes; vegetable waxes such as bayberry, candelilla, carnauba, rice bran oil and Japan wax; animal waxes such as beeswax and lanolin, and synthetic waxes such as polyethene waxes having a molecular weight of 2000-10000, and ethylene glycol diesters or triesters of long-chain fatty acids (C₁₈₋₃₆). Mixtures of waxes and oils can also be used for achieving desired properties as long as the melting point is 45° C. or more and preferably 55° C. or more.

According to yet another embodiment of the invention, the agent taking up and binding bacteria and proteins comprises a material which has been given positively charged functional groups by means, for example, of plasma treatment. In this treatment, nitrogen-containing groups, or other positively charged groups, can be supplied in gas form and bound to the surface of the material.

According to one embodiment of the invention, the agent for taking up and binding bacteria and proteins can comprise a coating of a polymeric material which has the ability to absorb bacteria. Such a coating can be produced by the surface of the material, in the wiping area, which is intended for wiping off being plasma-treated or corona-treated for the purpose of creating temporary negative charges. After that, the surface is sprayed with a positively charged polymer such as chitosan, polyetheneimine (PEI), polyallylamine hydrochloride (PAH) or some other suitable positively charged polymer.

Examples of positively charged polymers which can be used are

a) cationic or cationically modified polysaccharides such as cationic starch derivatives, cellulose derivatives, pectin, galactoglucomannan, chitin, chitosan or alginate,
b) a polyallylamine homopolymer or copolymer with or without modification units (suitable modification units of polyallylamines are disclosed, for example, in WO 00/31150), especially polyallylamine hydrochloride (PAH),
c) polyetheneimine (PEI),
d) a polyvinylamine homopolymer or copolymer with or without modification units,
e) poly(vinylpyrridine) or poly(vinylpyridinium salt) homopolymer or copolymer, including their N-alkyl derivatives,
f) polyvinylpyrrolidone homopolymer or copolymer, a polydiallyldialkyl, such as poly(N,N-diallyl-N,N-di-C₁-C₄-alkylammonium halide) as described in US 2004/0047979 A1, especially poly(N,N-diallyl-N,N-dimethylammonium chloride) (PDDA),
g) A homopolymer or copolymer of a quaternary di-C₁-C₄-alkyaminoethyl acrylate or methacrylate, for example a poly(2-hydroxy-3-methacryolylpropyl-tri-C₁-C₂-alkylammonium salt) homopolymer such as a poly(2-hydroxy-3-metacrylolylpropyltrimethylammonium chloride) or a quaternary poly(2-dimethylaminoethyl methacrylate or a quaternary poly(vinylpyrrolidone-co-2-dimethylaminoethyl methacrylate),
h) a poly(vinylbenzyl-tri-C₁-C₄-alkyammonium salt), for example a poly(vinylbenzyltrimethylammonium chloride),
i) polymers which are formed by reaction between ditertiary amines or secondary amines and dihaloalkanes, comprising a polymer of an aliphatic or araliphatic dihalide and an aliphatic N,N,N′,N′-tetra-C₁-C₄-alkylalkylenediamine, for example a polymer of a (a) propylene 1,3-dichloride or -dibromide or p-xylylene dichloride or dibromide and (b) N,N,N′,N′-tetramethyl-1,4-tetramethylenediamine,
j) POLYQUAD® as described in EP-A-456,467;
k) a polyaminoamide (PAMAM), for example a linear PAMAM or a PAMAM dendrimer such as an amino-terminated Starburst™ PAMAM dendrimer (Aldrich),
l) cationic acrylamide homopolymers or copolymers and their modification products, such as poly(acrylamide-co-diallyldimethylammonium chloride) or glyoxalacrylamide resins,
m) polymers formed by polymerization of N-(dialkylaminoalkyl)acrylamide monomers,
n) condensation products of dicyanodiamides, formaldehyde and ammonium salts,
o) typical wet strength agents which are used in paper manufacturing, such as urea formaldehyde resins, melamine formaldehyde resins, polyvinylamine, polyureide formaldehyde resins, glyoxal-acrylamide resins and cationic materials which are obtained by reaction between polyalkylenepolyamines and polysaccharides such as starch and various natural gums, as well as 3-hydroxyazetidinium ion-containing resins which are obtained by reacting nitrogen-containing compounds (for example ammonia, primary and secondary amines or N-containing polymers) with epichlorohydrin such as polyaminoamide epichlorohydrin resins, polyamine epichlorohydrin resins and aminopolymer epichlorohydrin resins as are mentioned, for example, in U.S. Pat. No. 3,998,690.

Preferred polycationic polymers are cationic or cationically modified polysaccharides such as starch or cellulose derivatives, chitin, chitosan or alginate, polyallylamine, homopolymer or copolymers, polyvinylamine homopolymers or copolymers or polyethyleneimine.

In order to improve the covering of the surface of the treated area, the surface can additionally be treated by means of LBL deposition (layer-by-layer deposition). The process involves nanoscale films being formed by alternately depositing monomolecular layers of two polymers which possess functional groups which can interact with each other, especially layers of cationic and anionic polymers, with the surface charge changing with each layer deposition. Such a multilayer coating can be brought about by spraying the treated area with a negatively charged polymer, after that spraying it with water and after that again spraying it with a positively charged polymer. The treatment is repeated until the desired number of layers has been obtained. Methods and suitable materials are described in detail in the international patent application PCT/EP 2005/010735.

The wiping area should be placed such that it interferes to the least possible degree with the absorption properties of the absorbent article. It has been found that a wiping area which extends for a distance, from the front edge towards the crotch portion, which corresponds to at most 25% of the length of the article between the first edge and the rear edge, and preferably at most 20% of the length, is sufficiently well separated from the crotch portion of the article.

In order to obtain a sufficiently large wiping surface, it is expedient for the wiping area to have an extent in the transverse direction of the article which corresponds to at least 40% of the length of the front edge. In the case of very small articles, for example diapers for infants, or in the case of especially narrow articles, it can be suitable for the wiping area to take up a larger part of the width of the article, that is of the length of the front edge. It is in general not a disadvantage for the wiping area to extend over the whole of the width of the article at the front edge.

Even if the wiping area can be arranged all the way out to the front edge, it is possible to conceive of embodiments in which the wiping area is arranged at a slight distance, for example 5-20 mm, from the front edge since some of the bacterium-binding and protein-binding agents which are used are effected by the high temperatures which occur during the manufacturing process when the absorbent article is welded or glued together at the edges. The effect of the temperature can cause the agents to lost the intended function or, as when using waxes, cause the agent to interfere negatively with the manufacturing process.

It can be advantageous for the wiping area to be identifiable visually and/or by tactile means such that the carer can determine directly the part of the absorbent article which is intended to be used for wiping. Such an indication can, for example, be brought about by the wiping area having a colour which differs from that of the outer layer of the article, a printed pattern, a text, a logo type or the like and/or having a differing surface structure or consisting of a separate component which is clearly distinguishable from the outer layer, such as a piece of material which is attached to the outer layer and which has discernible edges.

The wiping area can also exhibit an embossing pattern which, on the one hand, supplies a visual and tactile indicator and, on the other hand, provides an improved surface structure for mechanically removing faecal matter from the skin of the user. Other methods, such as creping, folding and perforating, can also be used to produce a surface which has improved mechanical wiping properties. However, it is important to ensure that the surface structure of the wiping area does not cause chafing or other discomfort for the user when the article is being worn. As an alternative, it is possible to arrange a removable protection layer over the wiping area.

One method of physically modifying the wiping area is to provide it with perforations. The perforations serve several purposes: on the one hand, they provide the wiping area with a surface structure which improves the mechanical wiping and, on the other hand, they allow any possible moisture and loose excrement to be absorbed into the product in connection with wiping. The perforations preferably occupy no more than 30% of the surface of the wiping area and have a diameter of 0.1-5 mm.

Other methods of improving the mechanical wiping properties of a wiping material for wiping off faecal matter are described in PCT/SE2004/000734.

### BRIEF DESCRIPTION OF THE FIGURES

The invention will be described in more detail below with reference to the figures which are shown on the attached drawings.

FIG. 1 shows a diaper having a wiping area in accordance with a first embodiment of the invention,

FIG. 2 shows a diaper having a wiping area in accordance with a second embodiment of the invention, and

FIG. 3 shows a diaper having a wiping area in accordance with a third embodiment of the invention.

### DESCRIPTION OF EXEMPLARY EMBODIMENTS

The invention relates to absorbent articles such as diapers and incontinence protection products.

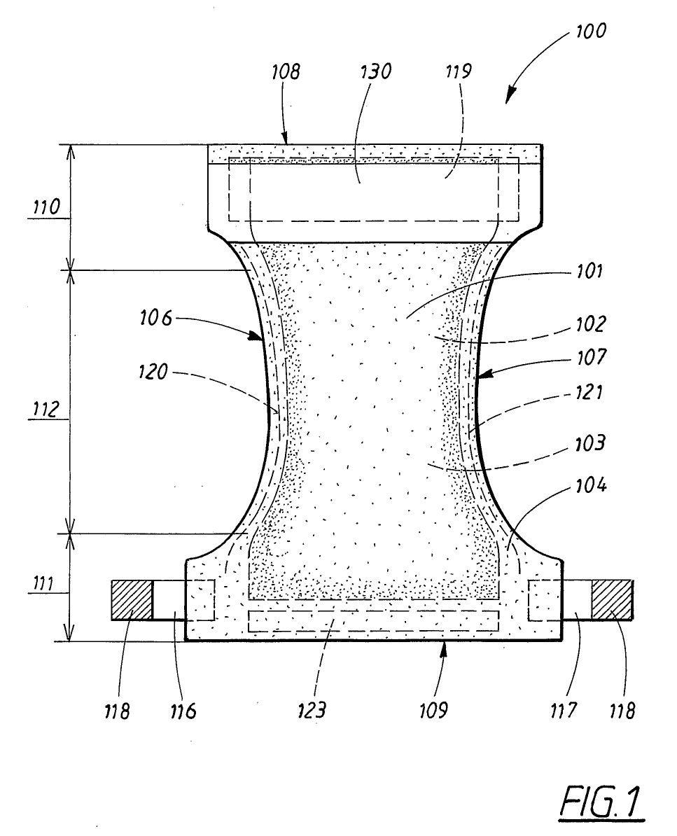

The diaper 100 in FIG. 1 is shown from the side which, during use, is intended to face the user. For the sake of clarity, the diaper is shown in the plane state with all the integral elastic components being extended. When in a state which is ready for use, i.e. when the elastic components are more or less contracted, the diaper is given a three-dimensional, curved form by the influence of the elastic members.

The diaper 100 comprises a liquid-permeable outer layer 101 and a liquid-impermeable outer layer 102 and also an absorption body 103 which is enclosed between the outer layers. The two outer layers 101, 102 have a greater extent in the plane than does the absorption body 103 and project out past the absorption body 103 around the whole of the periphery of the latter. The outer layers 101, 102 are connected to each other within the projecting parts 104, for example by means of gluing or welding with heat or ultrasound.

The liquid-permeable outer layer 101 can be composed of any material which is suitable for the purpose, such as a layer of nonwoven material, perforated plastic film, netting material, tow or the like. The outer layer 101 can naturally also consist of a laminate of two or more layers of the same or different materials. However, the liquid-permeable outer layer 101 does not need to be a separate component but can, instead, be an integrated part of the absorption body 103. In such an embodiment, the liquid-permeable outer layer 101 is not necessarily included in the projecting edge 104 around the absorption body 103. Examples of materials which can both be the outer layer 101 and be included in the absorption body 103 are foam, fibre wadding, nonwoven material or the like.

The liquid-impermeable outer layer 102 can consist of a liquid-impermeable plastic film, a nonwoven layer which has been coated with a liquid-barrier material or some other flexible material layer which has the ability to resist liquid penetration. However, it can be advantageous for the liquid-impermeable outer layer 102 to exhibit a certain breathability, i.e. to allow the passage of water vapour through the layer 102. The liquid-impermeable outer layer 102 can, like the liquid-permeable outer layer 101, be an integrated part of the absorption body and can, for example, consist of a liquid-impermeable skin-like surface on an absorbent foam material.

The diaper 100 is essentially hourglass-shaped, with side edges 106, 107 which extend in a longitudinal direction, a front edge 108 which extends in the transverse direction and a rear edge 109 which extends in the transverse direction, and exhibits a front waist portion 110, a rear waist portion 111 and a crotch portion 112 which is arranged between the front and rear waist portions. The front waist portion 110 is the part of the diaper 110 which is intended to face forwards on the user when the diaper is being used and the rear waist portion 111 is the part of the diaper which faces backwards on the user. The crotch portion 112 is the part of the diaper which, during use, is arranged in the crotch of the user and is expected to receive and absorb the body secretions which are delivered to the diaper 100. The crotch portion occupies approx. 50% of the length of the diaper and can be slightly displaced forwards or backwards in the diaper.

The diaper 100 is of the type which is fastened together during use such that, like underpants, it encloses the lower part of the trunk of the user. For this purpose, an attachment tab 116, 117 is arranged to project from each side edge 106, 107, by the rear edge 109 of the diaper. The attachment tabs 116, 117 are provided with attachment members 118 which are intended to interact with a reception area 119 which is arranged on the liquid-impermeable outer layer 102 on the front waist portion 110 of the diaper 100. When adhesive attachment members 118 are used, the reception area 119 expediently consists of some form of reinforcement of the liquid-impermeable outer layer 102, for example in the form of an additional plastic layer or a coating which is applied to the liquid-impermeable outer layer 102. When the attachment member 118 is a hook-and-loop member having interacting hook and loop members, the reception surface 119 is provided with loop members, for example in the form of a nonwoven material if the attachment tabs 116, 117 comprise hook members, and vice versa. Yet another alternative is for the diaper to be what is termed a pant diaper, which means that it is supplied to the user as a coherent absorbent panty. Another commonly occurring diaper type is fastened with the aid of a belt on which the diaper is fixedly or detachably suspended. The invention can be applied to any of these types of diapers and incontinence protection products.

The diaper 100 is furthermore provided with longitudinal elastic members 120, 121 which are fitted in a prestressed manner and which are applied along the side edges 106, 107 of the diaper. The elastic members 120, 121 contribute to curving the diaper 100 along the user's body during use and at the same time constitute the leg elastic of the diaper. In this way, the elastic members 120, 121 serve to hold the side edges 106, 107 of the diaper in sealing contact with the user's legs in order to counteract the possibility of any gaps, through which body liquid can leak out of the diaper, arising between the diaper and the user's body during use. In a corresponding manner, an elastic member 123 is arranged along the rear edge 109 and constitutes the waist elastic. It is naturally also possible to arrange waist elastic at the front edge 108 of the diaper 100. The elastic members can be in the form of one or more threads, ribbons or the like in a known manner.

The absorption body 103 can be constructed from one or more layers of absorbent material such as cellulose fluff pulp, tissue, absorbent foam, etc. It is also customary for the absorption body to contain superabsorbents, i.e. polymeric materials which can absorb body liquid corresponding to several times their own weight with the formation of a hydrogel. Such superabsorbents are usually present in the form of particles. However, fibres, flakes, granules and film are also met with. Furthermore, the absorption body 103 can include non-absorbing components such as stiffening elements, shaping elements, binders, etc. Different types of liquid-receiving porous structures such as fibre wadding or the like can also be included in the diaper 100.

A band-shaped wiping area 130 is arranged at the front waist portion 110 and extends in the transverse direction of the diaper 100 along the front edge 108 the whole way between the side edges 106, 107. The wiping area 130 is arranged at a slight distance from the front edge 108 and extends in the longitudinal direction of the diaper for a distance which corresponds to approx. 20% of the length of the diaper. The wiping area 130 is consequently arranged on the front waist portion 110 entirely outside the crotch area 112.

The wiping area 130 comprises an adsorptive agent which can take up and bind microorganisms and proteins. The adsorptive agent can be in the form of a coating, for example of the type which is described in the international patent application PCT/EP 2005/010735, or a coating of a wax having a melting point of at least 45° C. The adsorptive agent can also be in the form of a polymeric material which contains cationic groups. The wiping area 130 can be a part of the liquid-permeable outer layer 101 which has been coated with an adsorptive agent or has in some other way been imparted with a permanent modification of the surface which faces the user. Alternatively, the wiping area 130 can consist of a material other than the liquid-permeable outer layer 101. In the latter case, the wiping means can, for example, be a nonwoven material which is formed from the bacterium-adsorbing fibres which are described in WO 2004/062703.

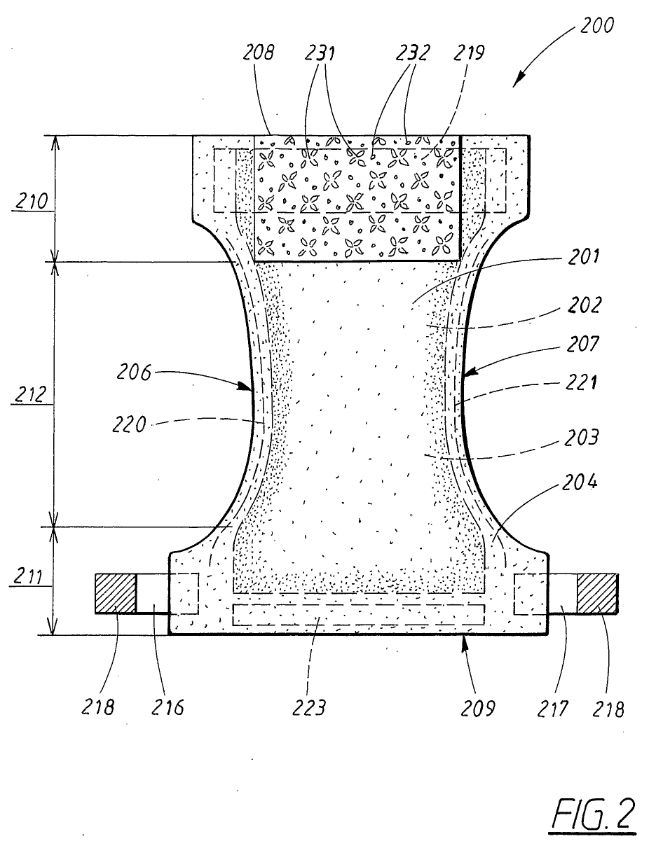

In FIG. 2, corresponding components have been numbered in analogy with the numbering in FIG. 1. The diaper in FIG. 2 only differs in the design of the wiping area 230 from the diaper which is shown in FIG. 1.

The diaper 200 in FIG. 2 consequently has a wiping area 230 which is arranged on the front waist portion 210 and extends, in the transverse direction of the diaper 100, along the front edge 208 for a distance which corresponds to just over half the length of the front edge 208. In the longitudinal direction of the diaper 200, the wiping area 230 extends from the front edge 208 for a distance corresponding to approx. 25% of the length of the diaper. Consequently, the wiping area 230 is arranged on the front waist portion 210, and entirely outside the crotch portion 212, in the exemplary embodiment which is shown in FIG. 2 as well. As has been previously mentioned, the extent of the wiping area 230 in the transverse direction of the diaper should in general be at least a third of the length of the front edge 208 in order to provide an adequate wiping surface. The wiping area 230 should not extend into the crotch portion 212 in the longitudinal direction of the diaper 200, which means that the wiping area 230 can only occupy at most 25% of the length of the diaper in the case of a diaper which has a crotch portion 212 which is placed symmetrically in the longitudinal direction. If the crotch portion 212 is displaced slightly forwards on the diaper, it is expedient for the wiping area 230 to occupy at most 20% of the length of the diaper.

The wiping area 230 in FIG. 2 exhibits an embossing pattern 231. Such an embossing pattern constitutes an indicator, which can be identified visually and in a tactile manner, of the location of the wiping area 230. Other methods for obtaining a visual indication of the location of the wiping area 230 are by means of using a coloured printed pattern or by means of the entire wiping area 230 being given a colour which differs from that of the liquid-permeable outer layer 201 of the diaper. It is naturally possible to combine these methods of marking where the wiping area 230 is located. The embossing pattern 231 which is shown should only be regarded as being one example. Any type of embossing pattern which is found on wiping materials can naturally be used for the wiping area 230.

The embossing pattern 231 also fulfils the additional function of improving the mechanical wiping properties of the wiping area 230 by means of creating an irregular, three-dimensional surface on the wiping area 230.

In addition to the embossing pattern 231, the wiping area 230 is provided with a plurality of perforations 232. While the embossing pattern 231 and the perforations 232 are shown here in combination, they can naturally be used separately if so desired. The perforations 232 also serve several purposes: on the one hand, they contribute to giving the wiping area 230 a surface structure which improves the mechanical wiping and, on the other hand, they allow any possible moisture and loose excrement to be absorbed into the product during wiping. The perforations preferably occupy no more than 30% of the surface of the wiping area and have a diameter of 0.1-5 mm.

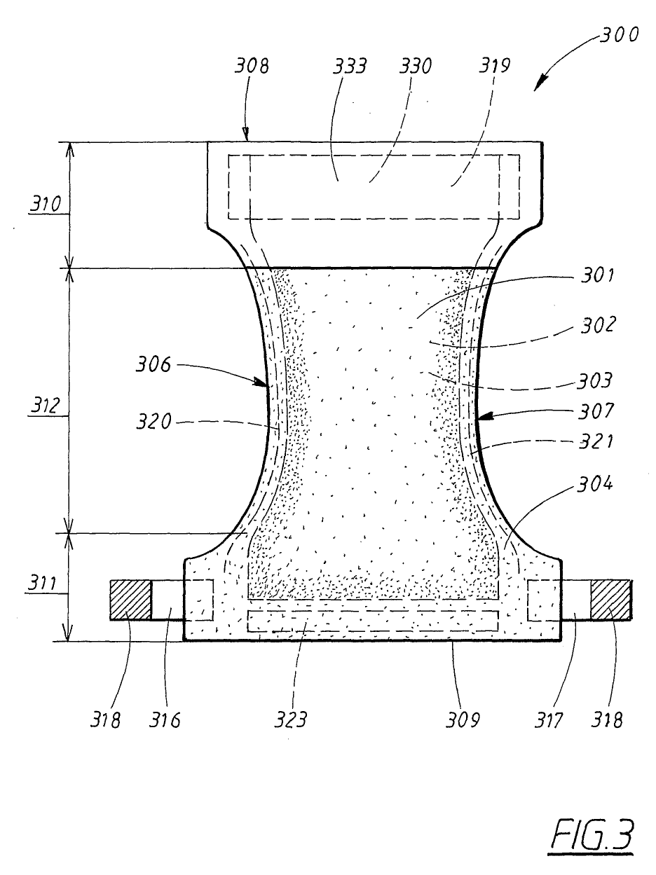

Yet another variant of a wiping area 330 is shown in FIG. 3.

In FIG. 3 as well, the only difference between the diaper 300 which is shown and the diaper 100 in FIG. 1 is that the wiping area 330 has a different design. The numbering of the different parts and components therefore corresponds to the numbering in FIGS. 1 and 2.

The wiping area 330 which is shown in FIG. 3 occupies the whole of the surface of the front waist portion 310 and consequently has the maximum size. The wiping area 330 is covered by a detachable protective layer 333 which is removed when the diaper 300 is to be used for wiping purposes. Such a protective layer 333 can be advantageous if the wiping area 330 has a surface which risks irritating the user's skin when the diaper 300 is being worn. The protective layer can also serve as a carrier for agents, such as lotions, oils or the like, which are to be transferred to the user's skin during use. Thin, soft nonwoven materials are suitable protective layers.

When a diaper or an incontinence protection product having a wiping area 130, 230, 330 in accordance with the invention is being changed, the carer can employ the used diaper or incontinence protection product which has just been removed from the user for wiping the genital area of the user before a new diaper or incontinence protection product is put on. In connection with this, the front waist portion of the diaper or incontinence protection product is grasped on the side which is orientated away from the user, i.e. on the liquid-impermeable outer layer in the examples shown, with the diaper or incontinence protection product being crumpled so that a good grip is obtained. After that, the drying is carried out using the crumpled diaper or incontinence protection product.

In many cases, when the quantity of faecal matter is not so large, or when the faecal matter is relatively dry and easy to collect, this wiping is sufficient and a new diaper or incontinence protection product can be put on without any further cleaning stage being required. The wiped-off skin surface both looks clean and is substantially less contaminated with bacteria than after wiping using a conventional absorbent article. An advantage is that the wiping reduces the quantity of bacteria on the skin appreciably without being bactericidal. This means that the normal, healthy bacterial flora on the skin is not disturbed. Yet another advantage is that the normal pH of the skin remains unaltered since no soap or other cleaning agent needs to be used.

The disclosure also offers a mild and effective way of reducing the quantity of microorganisms on a user's skin when changing absorbent articles which only contain urine, i.e. without having to use special cleaning agents or water.

The exemplary embodiments which have been described are only intended to illustrate the invention and are not to be regarded as limiting it. In particular, the different variants of wiping areas and adsorptive agents which have been described can be combined with each other and with types of diapers and incontinence protection products which are different from the all-in-one diaper which has been described. Consequently, the invention can be applied both to pant diapers and to belted diapers.

It is also possible to equip an absorbent article with more than one wiping area. In this connection, the wiping-off areas can be designed with surfaces which have different appearances or properties, in accordance with that which has been described above. It is furthermore also possible, if so desired, to arrange one or more wiping areas on the rear waist portion outside the crotch portion.

Naturally, the wiping area or wiping areas do not need to have the rectangular shape which has been shown but can, instead, be circular, trapezoidal or oval or be of any other suitable shape.

1. An absorbent article comprises: a longitudinal direction and a transverse direction, a front waist portion, a rear waist portion and a crotch portion which is arranged between the front and the rear waist portions,
a liquid-permeable outer layer and a liquid-impermeable outer layer and an absorption body which is enclosed between the outer layers,
a front edge which extends in the transverse direction and a rear edge which extends in the transverse direction,
a wiping area which is arranged on the liquid-permeable outer layer, wherein the wiping area being arranged outside the crotch area on the front waist portion and the wiping area comprises an adsorptive agent capable of taking up and binding microorganisms and proteins, the adsorptive agent being firmly united with the absorbent article.

2. The absorbent article according to claim 1, wherein the adsorptive agent comprises a polymeric material which has been grafted with positively charged groups or hydrophobic groups.

3. The absorbent article according to claim 2, wherein the polymeric material is a fibre material.

4. The absorbent article according to claim 1, wherein the adsorptive agent comprises a wax having a melting point which is higher than 45° C.

5. The absorbent article according to claim 1, wherein the adsorptive agent comprises a material which has been given positively charged functional groups by means of corona treatment or plasma treatment.

6. The absorbent article according to claim 1, wherein the adsorptive agent comprises a coating of a polymeric material which is able to bind bacteria.

7. The absorbent article according to claim 6, wherein the coating has been produced by means of LBL deposition (layer-by-layer deposition) and exhibits alternating layers of cationic and anionic polymers.

8. The absorbent article according to claim 1, wherein the wiping area extends, from the front edge towards the crotch portion, for a distance which corresponds to at most 25% of the length of the article between the front edge and the rear edge.

9. The absorbent article according to claim 1, wherein the wiping area has an extent in the transverse direction of the article which corresponds to at least 40% of the length of the front edge.

10. The absorbent article according to claim 1, wherein the wiping area can be identified visually.

11. The absorbent article according to claim 1, wherein the wiping area has an embossing pattern.

12. The absorbent article according to claim 1, wherein a detachable protective layer is arranged over the wiping area.

13. The absorbent article according to claim 1, wherein the wiping area is perforated.

14. The absorbent article according to claim 1, wherein the absorbent article has more than one wiping area.

15. The absorbent article according to claim 1, wherein the absorbent article is a diaper or an incontinence product.

16. The absorbent article according to claim 1, wherein the adsorptive agent comprises a polymeric material which has been grafted with positively charged groups and hydrophobic groups.

17. The absorbent article according to claim 1, wherein the adsorptive agent comprises a wax having a melting point which is higher than 55° C.

18. The absorbent article according to claim 1, wherein the wiping area extends, from the front edge towards the crotch portion, for a distance which corresponds to at most 20% of the length of the article between the front edge and the rear edge.

19. The absorbent article according to claim 8, wherein the wiping area has an extent in the transverse direction of the article which corresponds to at least 40% of the length of the front edge.
